(19) United States
(12) Patent Application Publication
(10) Pub. No.: US 2022/0288590 A1
(43) Pub. Date: Sep. 15, 2022

(54) MICROFABRICATED PARTICLE MANIPULATION DEVICE

(71) Applicant: Owl biomedical, Inc., Goleta, CA (US)

(72) Inventors: John S. FOSTER, Santa Barbara, CA (US); Mehran Hoonejani, Goleta, CA (US); Kevin SHIELDS, Santa Barbara, CA (US); Lily Li, Santa Barbara, CA (US)

(73) Assignee: Owl biomedical, Inc., Goleta, CA (US)

(21) Appl. No.: 17/831,445

(22) Filed: Jun. 3, 2022

Related U.S. Application Data
(63) Continuation-in-part of application No. 15/990,516, filed on May 25, 2018.
(60) Provisional application No. 62/645,508, filed on Mar. 20, 2018.

(51) Int. Cl. B01L 3/00; C12N 15/89; G01N 15/14

(52) U.S. Cl. CPC B01L 3/502761; C12N 15/89; B01L 3/502715; B01L 3/50273; G01N 15/1434; B01L 2200/027; B01L 2200/0652; B01L 2400/0487; B01L 2300/0672; B01L 2300/0663; G01N 2015/0065

(57) ABSTRACT

A microfabricated particle manipulation system, wherein a target particle is pierced by a microfabricated actuator or by a microfabricated knife edge. In either case, the particle membrane is altered, so as to allow material to traverse the membrane. The device may be used to extract cellular material from inside a cell, or to transfect a cell with foreign material.

### CROSS REFERENCE TO RELATED APPLICATIONS

This nonprovisional US Patent Application is a continuation-in-part (CIP), claims priority to U.S. patent application Ser. No. 15/990516, filed 25 May 2018, which in turn claims priority to U.S. Provisional Application Ser. No. 62/645,508 filed Mar. 20, 2018. These previous applications are incorporated by reference in their entireties.

### STATEMENT REGARDING FEDERALLY SPONSORED RESEARCH

Not applicable.

### STATEMENT REGARDING MICROFICHE APPENDIX

Not applicable.

### BACKGROUND

This invention relates to microelectromechanical systems (MEMS) devices. More particularly, this invention relates to a microfabricated particle manipulation device which can insert or extract material from biological cells or particles.

Transfection is a process whereby foreign genetic material is inserted into a target cell in order to alter, in some way, the function of the target cell. Transfection of animal cells typically is achieved by opening transient pores or “holes” in the cell membrane to allow the uptake of material. The holes may be created by squeezing or by applying an electric field, for example. Transfection can be carried out using calcium phosphate (i.e. tricalcium phosphate), by electroporation, by cell squeezing or by mixing a cationic lipid with the material to produce liposomes which fuse with the cell membrane and deposit their cargo inside.

Electroporation is a popular method whereby a transient increase in the permeability of a cell membrane is achieved when the cells are exposed to short pulses of an intense electric field. Calcium phosphate is again used, wherein a buffered saline solution (HeBS) containing phosphate ions is combined with a calcium chloride solution containing the DNA to be transfected. When the two are combined, a fine precipitate of the positively charged calcium and the negatively charged phosphate will form, binding the DNA to be transfected on its surface. The suspension of the precipitate is then added to the cells to be transfected (usually a cell culture grown in a monolayer). By a process not entirely understood, the cells take up some of the precipitate, and with it, the DNA. This process has been a preferred method of identifying many oncogenes.

In all the variations of performing transfection of cells, a substantial fraction of the starting cells do not achieve the desired transfection. Also, the cells that do achieve the transfection do not survive (are not viable, and subsequently die before being put to use). It is desirable to increase the efficiency of the transfection of cells, and also improve the viability of the resulting transfected cells.

In addition, it may be desirable to alter only certain specific cells, such as stem cells, cancer cells or T-cells, for example, in some way. However, these mentioned methods are, by their nature, batch processes, i.e. they are applied to large numbers of cells in solution, rather than to specific, targeted cells.

Accordingly, a device is needed that can transfect individual, targeted particles or cells, or a group or sample in a way that does not significantly damage the particles or cells.

### SUMMARY

Disclosed here is a method whereby cells may be transfected with foreign material, such as foreign genetic material. The method may be applied to a larger population of cells, and may alter any or all of these particles or cells. Alternatively, the method may use a system which identifies the target cells, for example, by laser-induced fluorescence, and applies the transfection process to those specific cells. A similar technique may be used to extract cellular material.

A microfabricated structure is designed to alter the membrane of a particle so as to allow a material to be placed within or extracted from the particle or cell. The alteration may be a piercing and/or a deformation of the cell membrane, which is sufficiently effective to allow the material to traverse the membrane and enter the cell. Accordingly, foreign material may be taken up by the nucleus of the cell, thus transfecting the cell. Alternatively, intracellular material may be extracted from the passing cells through the piercing or puncturing of the membrane. The alteration may be performed only on specific, targeted cells, or it may be applied to some or all of a population of particles or cells.

The microfabricated structure may be designed as a very small, sharp protuberance, such as a micro-scalpel or a needle. The cell membrane may be altered as a result of some or all cells flowing past the microfabricated structure, such that fluidic pressure is sufficient to allow the sharp protuberance to pierce the membrane. Alternatively, the cell may be forced against the protuberance by a narrow channel or by a sharp curve or corner in the microfabricated channel.

A plurality of embodiments is described herein, wherein the microfabricated particle manipulation system is formed on a substrate. In one embodiment, the microfabricated particle manipulation system may be formed on the substrate, and may include a microfabricated piercing structure fabricated on the substrate, and at least one microfabricated fluidic channel, wherein a fluid having particles suspended in the fluid flows within the at least one microfabricated fluidic channel, wherein the piercing structure pierces a membrane of at least some particles as the particles flow past the piercing structure. In another embodiment, the system may include an actuation mechanism fabricated on the substrate, and at least one microfabricated fluidic channel, wherein a fluid having target particles suspended in the fluid flows within the at least one microfabricated fluidic channel, wherein the actuation mechanism moves under an actuation force to press or puncture a target particle in the sample stream.

Accordingly, the alteration may be applied to either specific, target particles, or to some or all of a population or assembly of particles or cells.

These and other features and advantages are described in, or are apparent from, the following detailed description.

### BRIEF DESCRIPTION OF THE DRAWINGS

Various exemplary details are described with reference to the following figures, wherein:

FIG. 1 is a schematic cross-sectional illustration of an embodiment of a microfabricated particle transfection device;

FIGS. 2a, 2b are schematic, top down illustrations of embodiments of a piercing mechanism in a microfabricated particle transfection device;

FIGS. 3a, 3b are schematic, top down illustrations of other embodiments of a piercing mechanism a microfabricated particle transfection device;

FIG. 4 is a schematic, cross sectional illustration of other embodiments of a manipulation mechanism for a microfabricated particle transfection device;

FIG. 5 is a schematic illustration of another embodiment of a microfabricated particle transfection device, having an interrogation region to identify the particles, and a piercing structure and actuation mechanism;

FIG. 6 is a schematic illustration of another embodiment of a microfabricated particle transfection device, with a nano-scalpel and a micro-compression device;

FIG. 7 is a schematic illustration of another embodiment of a microfabricated particle transfection device, with a compressing structure and a micro-spike;

FIG. 8 is a schematic illustration of another embodiment of a microfabricated particle transfection device, with a compression channel and a stationary piercing structure;

FIG. 9 is a schematic illustration of another embodiment of a microfabricated particle transfection device, with a nano-scalpel coupled with positive injection or extraction;

FIG. 10 is a schematic illustration of another embodiment of a microfabricated particle transfection device, with a nano-scalpel coupled with positive injection or extraction;

FIG. 11 is a schematic illustration of another embodiment of a microfabricated particle transfection device which may push the particle toward the piercing mechanism; and

FIG. 12 is a schematic illustration of fluid focusing element usable with this system.

It should be understood that the drawings are not necessarily to scale, and that like numbers may refer to like features.

### DETAILED DESCRIPTION

The following discussion presents a plurality of exemplary embodiments of the novel particle manipulation system. The following reference numbers are used in the accompanying figures to refer to the following:

100, 110, 130 and 140 piercing/slicing mechanism/nano-scalpel

5 target particle

10 sample reservoir

15 optically etched hole

20 laser interrogation region

30 transfected output

40 foreign material reservoir

50 compression mechanism

55 microspike

60 corner piercing mechanism, dual nano-scalpel

200-1000 microfabricated particle manipulation embodiments

In some of the following embodiments of the systems and methods, a microfabricated piercing structure may pierce the membrane of a passing particle, for example, the membrane of a cell. The resulting damage to the membrane may be sufficient to allow material to pass into, and/or out of, the cell, thus altering the cell contents. If foreign material is added, the cell may be transfected. If material is removed, the cell may be functionally altered. The altered or transfected cell is then collected at an output.

For the transfection system, microfabricated fluidic channels in the microfabricated particle manipulation system may conduct a sample fluid between the input reservoir, the foreign material input, and the transfected output reservoir. The microfabricated fluidic channels are generally wider than the cell diameter. The sample fluid may contain a suspension of particles, including target cells and non-target material. The aim of the microfabricated particle manipulation system may be to transfect cells with foreign material, such as genetic fragments of RNA and DNA, organelles, proteins, nucleic acids, nucleotides and the like. The foreign material may include compounds not native to the target cell, that is, compounds not ordinarily found in an unaltered, unmanipulated cell. The foreign material may be stored in a reservoir or it may be included in the sample fluid. But in any case, the foreign material may be in fluid communication with the sample fluid in the microfabricated fluidic channel. Similar MEMS based systems may also be used to extract material from the interior of cells. Material (inserted or extracted) may be input with the sample or cell media or with a third port.

The structures shown in the accompanying figures and described below may be made using 3D MEMS lithographic processing technology, and fabrication methodologies which may be found in U.S. Pat. No. 9,372,144 (the '144 patent) issued 21 Jun. 2016 and incorporated by reference in its entirety. The particle manipulation system described here may also be used with MEMS cell sorting systems, such as those described in U.S. Pat. No. 9,194,786 (the '786 patent) issued 22 October Nov. 24, 2015 and also incorporated by reference in its entirety.

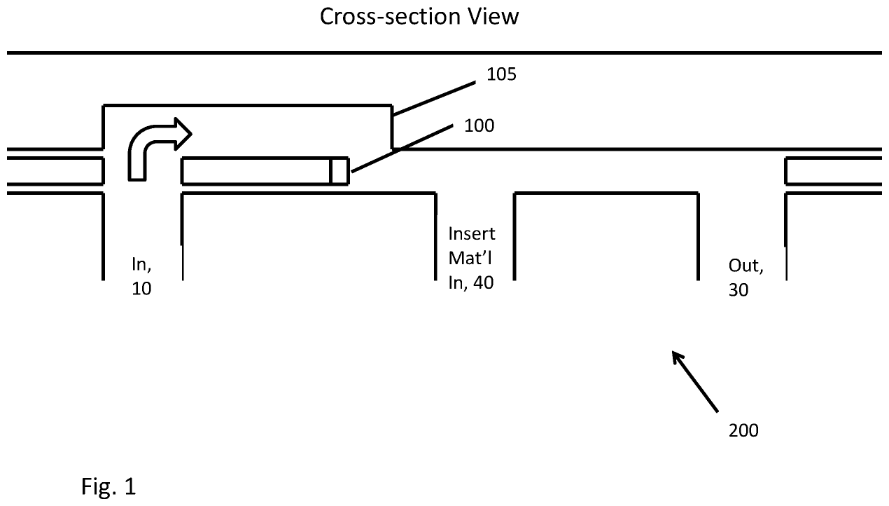

Advantages of the systems described here and illustrated by FIGS. 1-11 may include: It allows specific opening of cell membranes, which are repeatable and healable. The precise, sharp cutting may allow membranes to re-knit. It may be less traumatic than current discharge or electrophoresis, or focused radiation, or tearing with focused current from pillars, squeezing cells in sub-cell-sized-channels. The effectiveness or efficiency may be quite high, in terms of transfected cells/total cells processed. Because of its gentle nature, the viability of the transfected cells may remain quite high.

FIG. 1 is a schematic cross sectional illustration of a first embodiment of a microfabricated particle manipulation system 200. The device shown in FIG. 1 may use a piercing mechanism 100 to slice into cells as they flow within the microfabricated particle manipulation system 200. The sample fluid may flow from the input port 10 to the piercing mechanism 100. The thin membrane of the cells may be cut with the sharp MEMS feature 100. After being cut, sliced or pierced, the particle may flow past a source of foreign material 40. The foreign material 40 may enter the particle through the opening, cut, or puncture in the cell membrane. After transfection, the particle may exit through output channel 30.

The embodiment shown in FIG. 1 may be made with a three substrate stack, including a transparent glass layer, a middle layer having the actuator formed therein, and a third substrate having channels and ports formed therein. Further description of the fabrication process may be found in the '144 patent. The upper layer may be a glass layer wherein a 10 micron channel has been relieved to allow the passage of the particles or cells, using suitable etching techniques, such as dry or wet chemical etching through a mask. The knife edge or piercing structure 100 may be made in the actuator layer of the device described in the '144 patent. These features may be formed by deep reactive ion etching (DRIE) through a photolithographic mask. Such techniques are well known in the art.

At a point in the vicinity of the knife edge, the relieved area ends at the point labelled 105, forcing the passing particles or cells against the knife edge 100. The knife edge may rupture, slice or alter the cell membrane. A second port 40 may deliver foreign material such as oligonucleotides, into the area containing the ruptured cell. The tear or rupture may allow the foreign material to enter the cell or particle. Because of the microfabricated nature of the knife edge 100, it may be exceedingly sharp and narrow, and thus may cause a very clean cut with little trauma to the surrounding material. The membrane may then heal or re-knit, and the cell may remain largely undamaged and viable.

As will be described in further detail below, the piercing structure may have a plurality of sharp, knife-like or needle-like structures, which are fabricated lithographically to be exceedingly narrow and sharp. As a result, they may pierce the membranes easily, while causing relatively little damage or trauma to the particle or cell.

In the system shown in FIG. 1, and indeed in other embodiments that will be described below, the particles may be centered in the channel by inertial or viscous fluid forces, by non-linear flow, sheath flow, viscosity or by acoustic effects. This centering may ensure a collision with the MEMS knife, scalpel or piercing structure. This centering may be accomplished by a microfabricated fluidic manifold to focus the particles in a certain area within the fluid stream. The manifold may include a sample inlet and sheath fluid channel. The combined fluid may then flow around a focusing element coupled to the inlet channel, here a z-focusing channel, which tends to herd the particles into a particular plane within the flow. The combined fluid may then pass another intersection point, a “y-intersection point”, which introduces additional sheath fluid above and below the plane of particles. At the y-intersection point, two flows may join the z-focus channel from substantially antiparallel directions, and orthogonal to the z-focus channel. Alternatively, the device may use a spiral focusing channel such as described in U.S. patent application Ser. No. 14/919,786, (the '786 application) filed 22 Oct. 2015, and incorporated by reference in its entirety. This intersection may compress the plane of particles into a single point, substantially in the center of the stream.

Focusing the particles into a certain volume tends to decrease the uncertainly in their location, and thus the uncertainty in the timing. Such hydrodynamic focusing may therefore improve the speed and/or accuracy of the operation. Additional details relating to such hydrodynamic focusing may be found in the '144 patent. The degree of focusing and the location of the particles within the channel may affect the slicing force. Other techniques such and modifying the fluid density, viscosity, velocity may be used to control the hydrodynamic properties of the particles suspended in the fluid. These parameters may thus be used to control the precision or depth of the cutting for example.

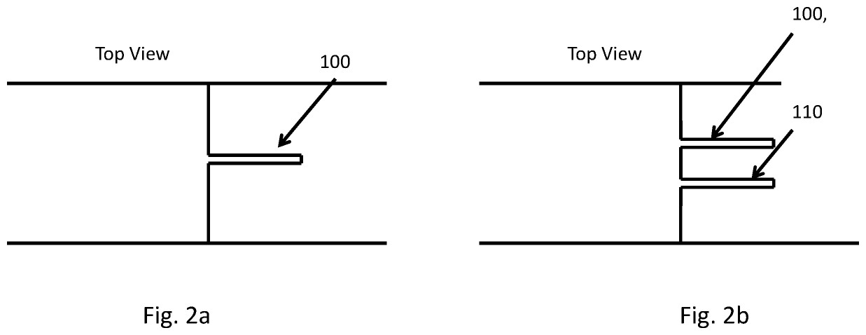

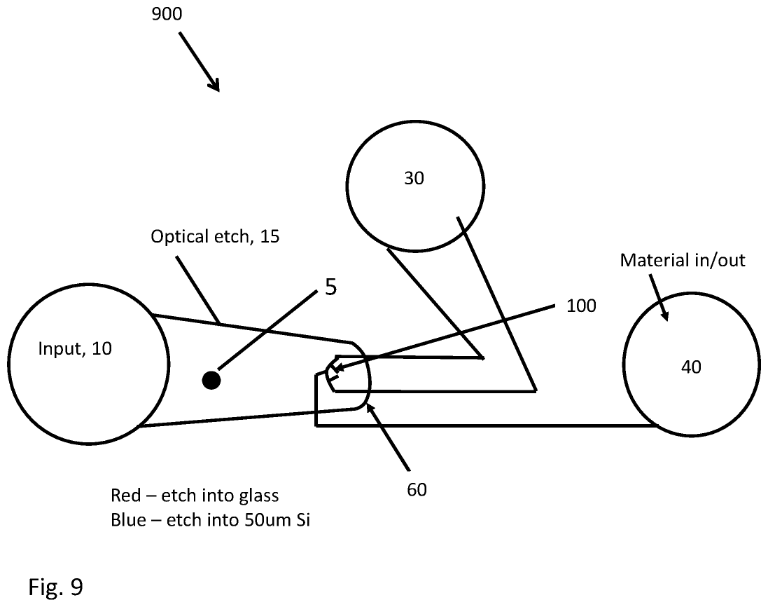

FIG. 2a, 2b are schematic illustrations of an embodiment of a piercing mechanism for a microfabricated particle manipulation device. FIG. 2a shows a top view of a single piercing structure 100, such as that shown in FIG. 1. FIG. 2b shows a top view of a plural piercing structure 100, 110, where multiple sharp edges 100, 110 may induce multiple cuts in the cell membrane. Such dual structures are described below and illustrated in FIGS. 9, 10 and 11. The features 100, 110 formed thereby may be very thin and sharp, e.g. 0.2 um wide, and sharp with a radius to <0.05 um.

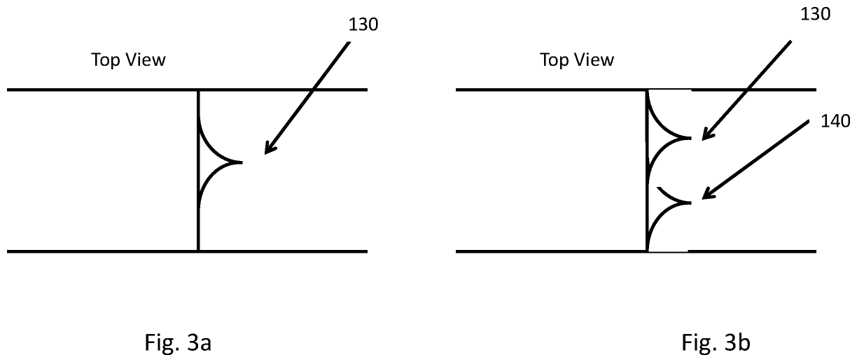

FIG. 3a, 3b are schematic illustrations of another embodiment of a piercing mechanism for a microfabricated particle manipulation device. FIG. 3a shows a top view of a single piercing structure 130, with a smooth, sloping lateral profile. FIG. 3b shows a top view of a plural piercing structure 130, 140, where multiple sharp edges 130, 140 with a smooth, sloping lateral profile, may induce multiple cuts in the cell membrane. The features 100, 110, 130 and 140 formed thereby may be very thin and sharp, e.g. 0.2 um wide, and sharp with a radius to <0.05 um, using photolithographic techniques.

The piercing structure 100 and 110, illustrated in FIGS. 2a and 2b and 130 and 140, illustrated in FIGS. 3a and 3b may be examples of a nano-scalpel for transfection. The term “nano-scalpel” and “micro-spike” are used herein to emphasize the small size of the piercing structure, and particularly its very sharp point. Because the point is lithographically defined, it may have a radius of curvature of less than 5 microns, and even well under 1 micron. The term “nano-scalpel” is not meant to imply that the structure necessarily has features on the nanometer scale. The piercing structures shown is FIGS. 3a and 3b may have such fine points, for example. Accordingly, the nano-scalpel, micro-spike or piercing structure may have a lithographically fabricated point with a radius of curvature of under 5 microns, and more particularly under 1 micron. Similarly, a “sharp edge” or an edge “configured to pierce a membrane” may also have a radius of curvature of less than about 10 microns along its cutting edge.

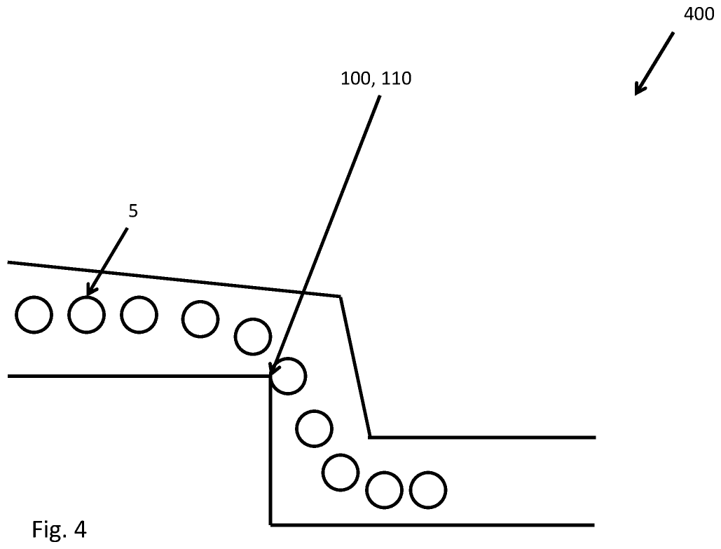

FIG. 4 is a schematic, cross sectional illustration of another embodiment of a microfabricated particle manipulation device 400. In this embodiment, the target particles 5 may be urged against the knife edge 100, 110 as the microchannel makes a turn. This turn may cause the particles to flow over the sharp edge as a result of the streamline in which they flow, having to make the turn shown.

The cells may be centered up-stream, using for example acoustic centering, non-linear flow, sheath flow, viscosity, etc. as described above. The nano-scalpel architecture can be similar to or the same as valve-type, cell sorting chips as disclosed in, for example, the '144 patent. The top layer may be a transparent material such as glass, with a recessed etch and covering a silicon layer wherein the actuator is formed. An actuator, wedge, needle or scalpel 100 may be formed in the silicon using deep reactive ion etching, for example.

The particular cell path as illustrated in FIG. 4 may be determined by the detailed shape of flow channels. Proper design of these channels may result in the guiding of cells past and over the scalpel even with the channels somewhat larger than cells. By modifying the density and/or the viscosity and visco-elasticity of the buffer fluid used to control the amount and/or depth of slicing (e.g. the corner cutting of the cell may be driven by the viscosity of the buffer fluid and by the density of the cells versus the buffer).

The embodiment shown in FIG. 4 may also make use of a transient pressure pulse produced by an actively controlled actuator. Designs for such microfabricated actuators may be found in U.S. patent application Ser. No. 15/436,771, filed 18 Feb. 2017 and incorporated by reference in its entirety. The actuator may produce a positive pressure pulse in a fluid containing the foreign material for insertion into the altered cell membrane. This mechanism may therefore assist in the effectiveness of the transfection.

This transient pressure pulse may also be used to urge the particle or cell against the piercing structure, which may make the piercing structure more effective.

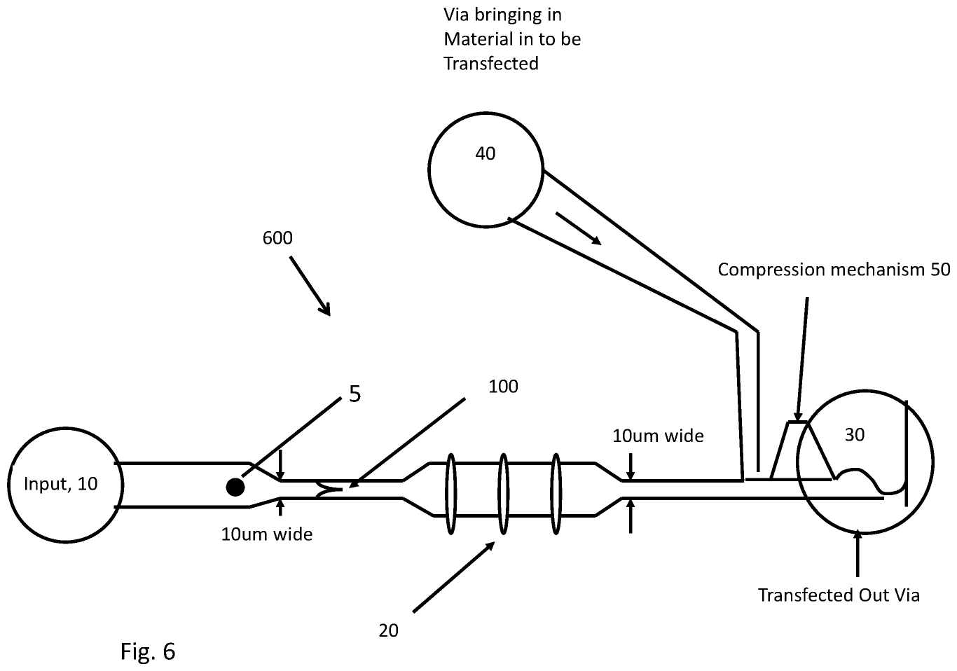

Alternatively, the pressure may be exerted actively on the cell by a compression mechanism described in greater detail below. This compression mechanism may widen or expand the cut formed in the membrane, and thereby assist in the uptake of the foreign material. Similarly, the actively controlled compression mechanism may also be provided with a piercing structure, such that the membrane tear is only applied to certain identified and targeted particles or cells, as illustrated by FIGS. 6, 7 and 8.

In any case, these microfabricated mechanisms are likely to be gentler, applying only very limited and targeted damage, such that the viability of the cell remains high, as does the transfection rate.

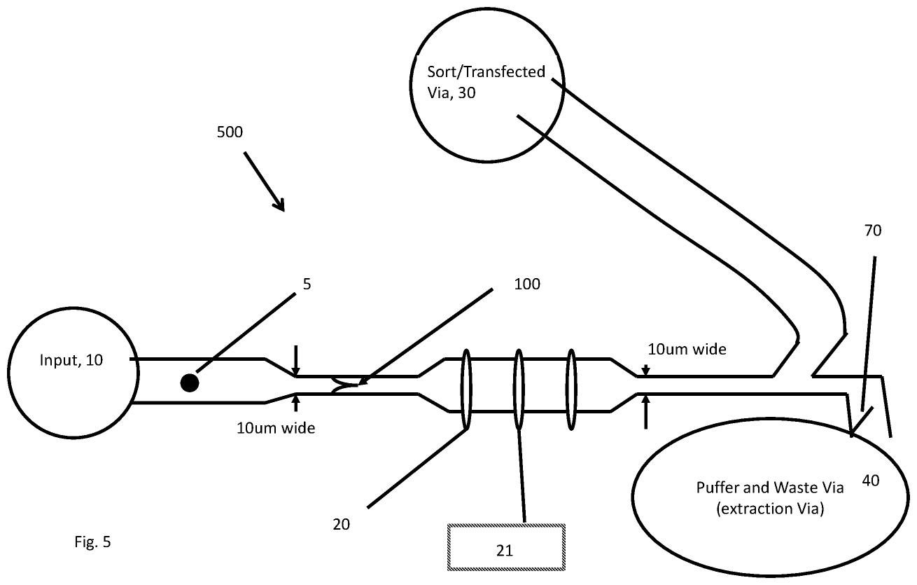

FIG. 5 is a schematic illustration of another embodiment of a microfabricated particle manipulation device 500. FIG. 5 may illustrate a nano-scalpel coupled with positive injection or extraction. The scalpel piercing structure 100 may be of similar design as was shown in FIG. 3a, 3b, for example. The cells may be pierced as they pass by the piercing structure 100. As in the other embodiments, a laser interrogation scheme 20 may identify the proper target cell, 5. When a target cell is identified, a positive pressure source 70, acting on a reservoir containing the foreign material 40 may be actuated, pulsed or puffed, sending a volume of the foreign material into the channel in the vicinity of the pierced cell, which then incorporates the foreign material from the puffer source 40. The transfected cells are then collected in the transfected cell reservoir 30.

The actuator 70 shown in the puffer/foreign material region 40 may also be actuated in the opposite sense, applying negative pressure to the cell and thus extracting material from the interior of the cell. The extracted material may proceed into the extraction via 40.

FIG. 6 is a schematic illustration of another embodiment of a microfabricated particle manipulation device 600. FIG. 6 may illustrate a nano-scalpel transfection mechanism using micro-compression device 50. The scalpel piercing structure 100 may be of similar design as was shown in FIG. 3a, for example. Once again, the laser interrogation regions 20 may identify the proper target cell, 5. Positive pressure from a compression mechanism 50 may deform the cell 5 inside the channel. It should be understood that the compression mechanism 50 may be used with, or without, the piercing mechanism, and that the piercing mechanism 100 may be used with, or without, the compression mechanism 50. In any case, the compression mechanism 50 may create positive pressure to pump the surrounding foreign material into the target cell 5. The foreign material may be stored in a reservoir 40, and released upon detection of a target cell 5 within the channel. The compression mechanism 50 may be magnetically actuated, such as is described in U.S. Pat. No. 9,404,838 (the '838 patent) issued 2 Aug. 2016, and incorporated by reference in its entirety.

The system may be triggered by the laser interrogation 20, and a computer may then actuate compression mechanism 50. The compression mechanism may also be used to catch, trap or temporarily immobilize a target cell 5. The transfected cells may go vertically down into transfected out via 30. In some embodiments, the laser interrogation region 20 may include a microprocessor 21, computer or controller, which analyzes signals from the laser interrogation region, and uses artificial intelligence or machine learning to better identify the target particles flowing in the sample stream.

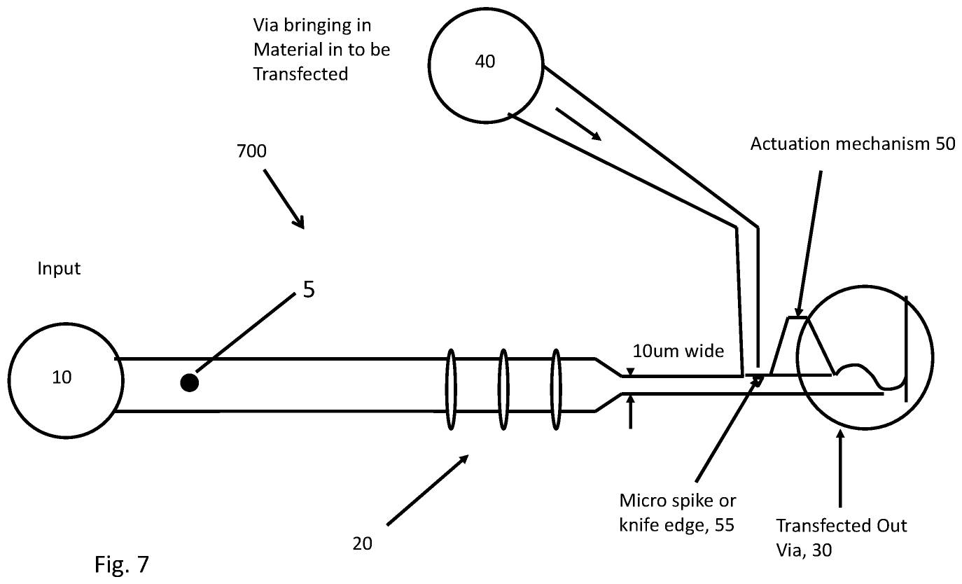

FIG. 7 is a schematic illustration of another embodiment of a microfabricated particle manipulation device 700. FIG. 7 may illustrate a compressing structure 50 which is equipped with a micro-spike 55. This embodiment may not have an upstream stationary scalpel piercing structure 100. Accordingly, embodiment 700 may only act on specific, targeted particles or cells. Once again, the laser interrogation regions 20 may identify the proper target cell, 5.

As before, the system may be triggered by the laser interrogation 20, and a computer may then actuate compression mechanism 50. The compression mechanism 50 may also be used to catch, trap or temporarily immobilize a target cell 5. The transfected cells 5 may go vertically down into transfected out via 30.

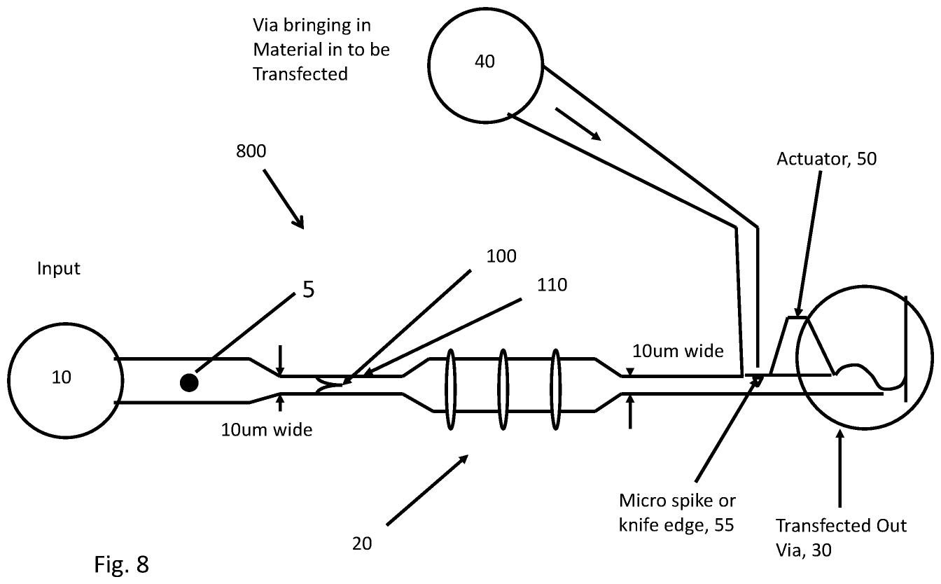

FIG. 8 is a schematic illustration of another embodiment of a microfabricated particle manipulation device 800. FIG. 8 may include a compression mechanism 50, a stationary piercing structure 100. Embodiment 800 may also include an active, actuated compression structure 50 which is equipped with a micro-spike 55. Accordingly, the compression structure may also have a piercing structure formed thereon. Accordingly, embodiment 800 may have both an upstream, stationary, scalpel-like piercing structure 100 (may be of similar design as was shown in FIG. 3a, for example) as well as an active, actuated compression mechanism 50 (similar to that illustrated in FIG. 7). Once again, the laser interrogation regions 20 may identify the proper target cell, 5 for the active, actuated compression mechanism 50. The passive, stationary scalpel 100 may act on some or all of the passing particles, whereas the active, actuated compression mechanism 50 may act only on the particle identified by the laser interrogation structure 20.

FIG. 8 illustrates this variant in some detail. The cell compression structure 50, may include a sharp, pointed micro-spike 55 on its movable portion. Once again, it should be understood that the compression mechanism 50 and micro-spike 55 may be used with, or without, the piercing mechanism 100, and that the piercing mechanism 100 may be used with, or without, the compression mechanism 50 and micro-spike 55.

Using the micro-spike 55, the compression mechanism 50 may not only deform the target cell 5, it may also be used to pierce the cell membrane with micro-spike 55. However, this structure may also have the compression channel with stationary piercing structure 100. The stationary piercing structure 100 may act on some or all of the passing particles or cells, whereas the actuated compression mechanism 50 may act on targeted particles or cells alone.

FIG. 9 is a schematic illustration of another embodiment 900 of a microfabricated particle transfection device. FIG. 9 may illustrate a nano-scalpel coupled with positive injection or extraction. FIG. 9 may include a vertical channel 15 etched into a transparent glass layer similar to FIG. 1. At the corner of the vertical channel, the particles or cells pass over the piercing structure 100 in the region of the corner 60. The scalpel piercing structure 100 may be of similar design as was shown in FIG. 3b, for example, but in the case of FIG. 9, the scalpel may be formed with two narrow beams both positioned in a corner 60 (similar to a can opener) as shown qualitatively in FIG. 3b.

In this embodiment, there may be no laser interrogation region, such that particular particles are not identified for special treatment. Accordingly, the piercing structure is applied to some or all of the particles in the sample without distinction. The foreign material in passage 40 is thus applied to all particles or cells without distinction.

Positive pressure at material via 40 may inflate cell with material (in buffer fluid), sends into transfected via output 30. Negative pressure at material via 40 may deflate the cell and extract material, and the extracted material may proceed into extraction via 30. Because of the precision and sharpness of the microfabricated piercing structures, the possibility exists to extract material from the cells, without causing enough damage to kill the cells.

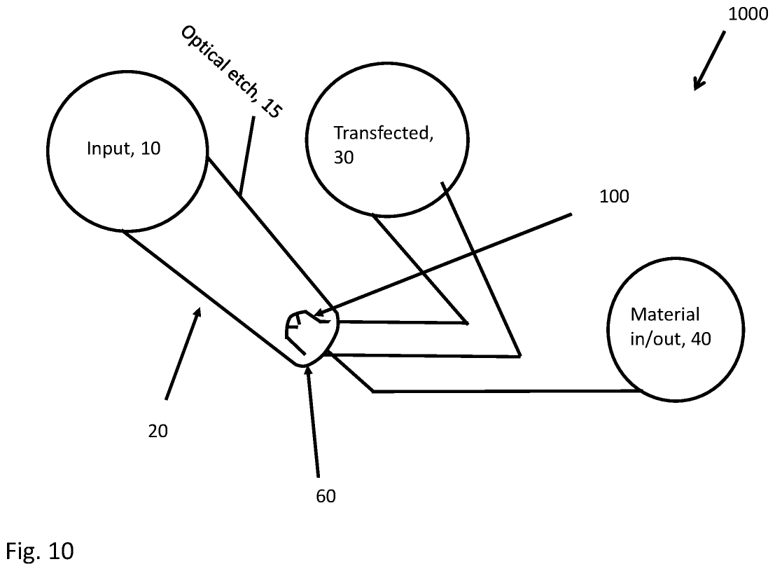

FIG. 10 is a schematic illustration of an embodiment of a microfabricated particle manipulation device 1000. FIG. 10 may illustrate a corner 60 located nano-scalpel 100 coupled with positive injection or extraction, and using also a laser interrogation 20. In the case of the embodiment shown in FIG. 10, there may also be fluid flow provided which may push the cells toward the piercing mechanism 100.

In this embodiment, the piercing mechanism 100 is a passive knife edge, and is therefore applied to some or all of the particles or cells in the sample without distinction. However, the laser interrogation 20 may be used to determine the timing of the release of the foreign material into the channel for transfection by the opened particles or cells.

The scalpel piercing structure 100 may be of similar design as was shown in FIG. 2a, for example, but in the case of FIG. 10, the scalpel may be formed with two narrow beams 60 (similar to a can opener) as was shown qualitatively in either FIG. 2b or 3b. The structure labeled “optical etch” may be a hole etched in a glass layer, and thus into the paper of FIG. 10. It appearance in FIG. 10 is a perspective view of this hole, rendered on flat paper.

Once again, the laser interrogation region 20 may identify the proper target cell, 5, for manipulation by the piercing structure 100/60 and transfection with the foreign material in 40. In this case, a transient pressure pulse may be emitted from the foreign material reservoir 40 as the lanced target particle passes. Alternatively, there may be no laser interrogation region, and the piercing structure alters some or all of the passing particles or cells. Accordingly, as in all embodiments, it should be understood that a compression mechanism 50 (as shown in FIG. 6) and may be used with, or without, the piercing mechanism 60, and that the piercing mechanism 60 may be used with, or without, the compression mechanism 50. These embodiments may, in turn, be used with or without a laser interrogation region 20.

Positive pressure at material via 40 may inflate the cell with material, included with the buffer, for example, and sends the particle or cell into the transfected via output 30. The positive pressure may also help force the cells against the scalpel 100, increasing the possibility of the cells being cut by the scalpel 100.

Negative pressure at material via 40 may deflate the cell and the extracted material proceeds into extraction via 30. Negative pressure at via 40 may also force the cells against the scalpel increasing the possibility of cells being cut by it.

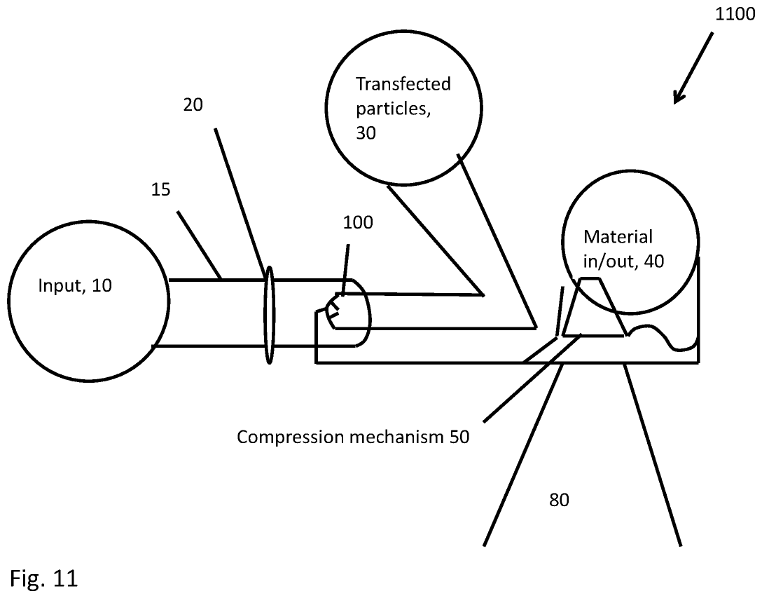

FIG. 11 is a schematic, top down illustration of another embodiment of a microfabricated particle transfection device 1100. In the case of the embodiment shown in FIG. 11, there may also be fluid flow provided which may push the cells toward the piercing mechanism 100. In addition, there may also be provided a compression mechanism 50 similar to that shown in FIGS. 6 and 7.

The scalpel piercing structure 100 may be of similar design as was shown in FIGS. 3a and 3b, for example, but in the case of FIG. 11, the scalpel may be formed with two narrow beams 60.

Once again, the laser interrogation regions 20 may identify the proper target cell 5. Also, it should be understood that a compression mechanism 50 (as shown in FIG. 6) may be used with, or without, the piercing mechanism 60, and that the piercing mechanism 60 may be used with, or without, the compression mechanism 50. The structure labeled “optical etch” may be a hole etched in a glass layer, and thus into the paper of FIG. 11.

Positive pressure at material via 40 may inflate a cell with material (in buffer fluid), and in conjunction with the compression mechanism 60, may help the cells take up the foreign material from the source 40. The transfected target particles 5 will then be sent into transfected via output 30. The positive pressure may also help force the cells against the piercing structure 100, increasing the possibility of the cells being cut by the scalpel 100. Negative pressure at material via 40 may deflate the cell and the extracted material proceeds into extraction via 30. Negative pressure may also force the cells against the scalpel 100 increasing the possibility of cells being cut by it.

Accordingly, a microfabricated particle manipulation system is described which may be formed on a substrate that manipulates particles in a sample stream. The system may include a microfabricated piercing structure fabricated on the substrate, having at least one edge configured to pierce a cell membrane, at least one microfabricated fluidic channel, wherein a fluid having target particles suspended in the fluid flows within the at least one microfabricated fluidic channel, wherein the piercing structure pierces a membrane of the target particle as the target particle flows past the piercing structure.

In other embodiments, the system may alternatively include an interrogation region that distinguishes a target particle suspended in the sample stream flowing within the microfabricated fluidic channel and an actuation mechanism fabricated on the substrate and shaped to exert a force within the microfabricated fluid channel. Foreign material may be provided in communication with the microfabricated fluidic channel, wherein the foreign material includes compounds not native to the target cell, and at least one microfabricated fluidic channel, wherein a fluid having target particles suspended in the fluid flows within the at least one microfabricated fluidic channel, wherein the actuation mechanism moves under an actuation force to deform a target particle in the sample stream, deforming the target particle, allowing foreign material to enter or exit the particle the particle through the pierced membrane.

In the system, the foreign material is at least one of DNA, RNA, a biologically active compound and a chemically active compound, and may be stored in a reservoir. The foreign material may be ejected from the reservoir by a transient positive pressure pulse into the microfabricated channel in the vicinity of the target particle. The foreign material may be ejected into the channel only when the target particle is present in the microfabricated channel.

The manipulation system may also apply a positive fluid pressure into the target particle, inflating the target particle. The manipulation system may also apply a negative fluid pressure into the target particle, deforming or deflating the target particle. The manipulation system may withdraw material from the interior of the target particle. The manipulation system may also apply a positive fluid pressure into the target particle, deforming or inflating the target particle. The piercing structure may comprise a knife edge, sufficiently sharp to cut a membrane of the target particle. The manipulation system may comprise a plurality of knife edges, which cuts a membrane of the target particle. The piercing structure comprises one or more sharp edges, which together may slice a target particle to open a membrane surrounding the particle. Accordingly, the system may use a microfabricated knife edge to slice through the cell membrane, as the cell flows past the knife edge in the microfluidic channel. The microfabricated fluidic channel may also comprise a barrier or narrowed portion, wherein this barrier or narrowed portion urges the target cell onto the knife edge to be sliced as it flows past, and wherein the target cell is never stationary with respect to the knife edge.

In another embodiment, the knife edge may be fabricated on the actuator, and may thereby be movable. In this embodiment, the alteration of the target cell may be made specifically, that is, the cell membrane of a particular target cell may be pierced, sliced or manipulated, and only the target cell. As illustrated in FIGS. 7 and 8. A micro spike, knife edge or piercing structure 55 may be placed on an actuation mechanism 50. The micro spike, knife edge or piercing structure may thereby be movable with respect to at least one microfabricated fluidic channel. The actuation mechanism 50 may be electromagnetic as described elsewhere, or it may use some other actuation mechanism such as electrostatic. The actuation mechanism 50 may be coupled to the controller which may direct the actuation mechanism to move based on the results of the laser interrogation region, 20. Using this embodiment, the slicing, piercing and transfection may be applied to a specific target particle or cell. As identified by the tag conjugated to a specific antigen expressed on the surface of the cell.

Using this embodiment, various genetic therapies or gene altering techniques may be applied to specific target cells.

The controller 21 may be programmed to execute various machine learning or artificial intelligence algorithms which may improve the performance of the system. This artificial intelligence mechanisms may improve the precision of the identification of the target cell identification. Such techniques are described more fully in co-pending U.S. patent application Ser. No. 17/716007, filed Apr. 8, 2022.

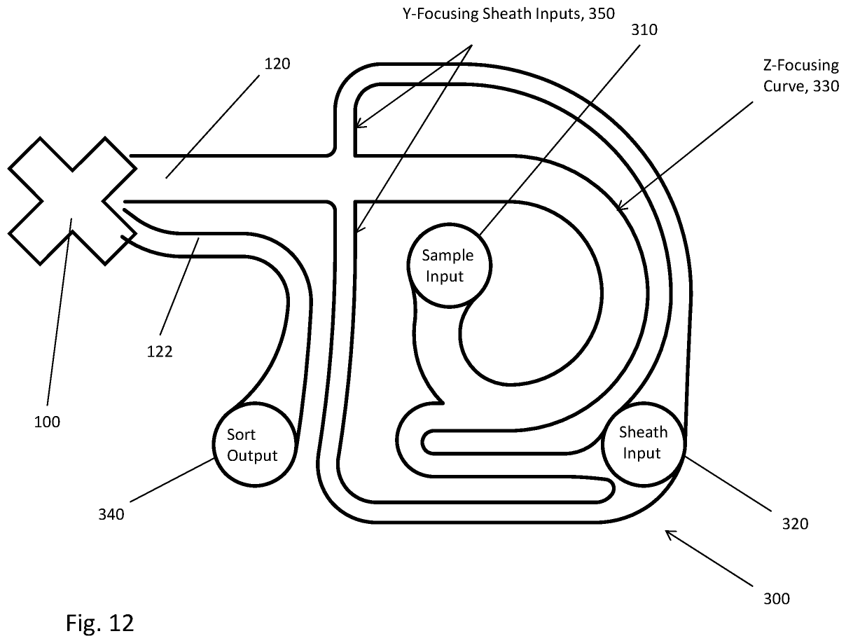

The performance of the system may be further improved by the use of a fluid focusing element, 300. This fluid focusing element 300 may be illustrated in FIG. 12. FIG. 12 depicts the microfabricated focusing element 300 which may be used to focus the particles in a certain area within the fluid stream. As the name suggests, the sheath fluid inlet channel 320 adds a sheath fluid to the sample stream, which is a buffering fluid which tends to dilute the flow of particles in the stream and locate them in a particular portion of the stream. The combined fluid then flows around a focusing element 300 coupled to the sample inlet channel 120. The focusing element 300 may include here a z-focusing curve 330, which tends to herd the particles into a particular plane within the flow. This plane is substantially in the plane of the paper of FIG. 8. The combined fluid in the focusing element 300 then passes another intersection point, a “y-intersection point” 350, which introduces additional sheath fluid above and below the plane of particles. At the y-intersection point 350, two flows may join the z-focus channel 330 from substantially antiparallel directions, and orthogonal to the z-focus channel 330. This intersection may compress the plane of particles into a single point, substantially in the center of the stream. Accordingly, at the y-intersection point 350 the target particles may be compressed from a plane to a stream line near the center of the z-focus channel 330 and sample inlet channel 120. Focusing the particles into a certain volume tends to decrease the uncertainly in their location, and thus the uncertainty in the timing of the opening and closing of the movable member or valve 110. Such hydrodynamic focusing may therefore improve the speed and/or accuracy of the sorting operation and the slicing operation.

In one exemplary embodiment of the microfabricated particle manipulation device 100 with hydrodynamic focusing illustrated in FIG. 8, the angular sweep of z-bend 330 is a curved arc of about 180 degrees. That is, the approximate angular sweep between the junction of the sheath fluid inlet channel 320 and the y-intersection point 350, may be about 180 degrees. Generally, the radius of curvature of the z-bend 330 may be at least about 100 microns and less than about 500 microns, and the characteristic dimension, that is the width, of the channels is typically about 50 microns to provide the focusing effect. In one embodiment, the radius of curvature of the channel may be about 250 microns, and the channel widths, or characteristic dimensions, for the sample inlet channel 120 and z-bend channel 330 are on the order of about 50 microns. These characteristic dimensions may provide a curvature sufficient to focus the particles, such that they tend to be confined to the plane of the paper upon exit from the z-focus channel 330 at y-intersection point 350. This plane is then compressed to a point in the channel at the y-intersection point 350. Accordingly, the y-intersection 350 flows along with the z-focusing element 330 may urge the particles into a single stream line near the center of the microfabricated sample inlet channel 120.

Further details of alternative designs are set forth in co-pending U.S. patent application Ser. No. 16/933,149, filed Jul. 20, 2020, and incorporated by reference. The fluid focusing element 300 may be a passive device which tends to urge the particles in the sample stream to be concentrated in a particular streamline, for example in the middle of a microfabricated channel. The device may use hydrodynamic effects to accomplish this target cell focusing. Better, more repeatable positioning of the target particles make the estimation of the velocity with which these particles are flowing in the stream more accurate. This may make the manipulation of particular target particles more accurate, and especially when coupled with the machine learning or artificial intelligence algorithms described above.

In the systems, the actuator may force material out of the interior of the target particle as a result of the deformation. The system may further comprise a fluidic focusing element, which tends to concentrate the particles toward the center of the microfabricated channel. The actuator may be formed in a plane parallel to a top surface of the substrate and moves in that plane when actuated.

In the systems, when the material enters in a sample channel, and the material may enter the particle through a hole in the sliced membrane of the target particle.

The system may comprise a source of positive and negative pressure, wherein the positive pressure may force a foreign material into a target cell and the negative pressure may extract material from an interior of the target cell. It may further comprise a compression structure and a piercing structure. The compression structure may be magnetically actuated. The compressing structure may also have a piercing structure formed thereon.

The microfabricated particle manipulation system may manipulate particles in a sample stream, wherein passive manipulation is applied to the particles without identification (no interrogation, passive piercing).

In one embodiment. a microfabricated particle manipulation system may be formed on a substrate that manipulates particles in a sample stream. The system may comprise a laser interrogation region that identifies target particles and applied the manipulation to the target particles, wherein passive manipulation is applied to the particles without identification (laser interrogation).

In one embodiment, a microfabricated particle manipulation system may use a laser interrogation region that identifies target particles, a manipulation stage that manipulates the target particles by piercing a membrane of the particles, a transient pressure generator that supplies a foreign material to the manipulated cells at a time determined by the laser interrogation region.

In one embodiment, a microfabricated particle manipulation system may use a laser interrogation region that identifies target particles, an active, actuated particle manipulation stage that alters a membrane on target particles, and a transient pressure generator that supplies a foreign material to the manipulated cells at a time determined by the laser interrogation region.

While various details have been described in conjunction with the exemplary implementations outlined above, various alternatives, modifications, variations, improvements, and/or substantial equivalents, whether known or that are or may be presently unforeseen, may become apparent upon reviewing the foregoing disclosure. Accordingly, the exemplary implementations set forth above, are intended to be illustrative, not limiting.

What is claimed is:

1. A microfabricated particle manipulation system formed on a substrate, that manipulates particles in a sample stream, comprising:
at least one microfabricated fluidic channel;
a microfabricated knife edge structure fabricated on the substrate and disposed within the fluidic channel, having at least one knife edge configured to slice a cell membrane, as the cell travels past the knife edge;
a fluid having target particles suspended in the fluid, the fluid flowing within the at least one microfabricated fluidic channel;
a laser interrogation region that distinguishes a target particle suspended in the sample stream flowing within the microfabricated fluidic channel, based on its fluorescent response to laser radiation; and
foreign material in communication with the microfabricated fluidic channel, wherein the foreign material includes compounds not native to the target particle and wherein the foreign material is release after the target cell is distinguished in the laser interrogation region;

2. The microfabricated particle manipulation system of claim 1, wherein the knife edge is fabricated on an actuation mechanism, and is thereby movable relative to the at least one microfabricated fluidic channel.

3. The microfabricated particle manipulation system of claim 2, wherein the foreign material is disposed in a reservoir, and comprises at least one of DNA, RNA, organelles, proteins, nucleic acids, nucleotides, a biologically active compound and a chemically active compound

4. The microfabricated particle manipulation system of claim 3, wherein the foreign material is ejected from the reservoir by a transient positive pressure pulse into the microfabricated channel in the vicinity of the target particle.

5. The microfabricated particle manipulation system of claim 4, wherein the foreign material is ejected into the channel only when the target particle is present in the microfabricated channel.

6. The microfabricated particle manipulation system of claim 2, wherein the microfabricated knife edge structure is configured to slice pierce or deform the target particle, as identified by the laser interrogation region..

7. The microfabricated particle manipulation system of claim 6, further comprising a controller which is programmed to activate the knife edge upon identification of a particular target particle.

8. The microfabricated particle manipulation system of claim 7, wherein Controller is further programmed to apply artificial intelligence techniques to the identification of the particulate target particle.

9. The microfabricated particle manipulation system of claim 6, wherein the microfabricated knife edge structure is configured to insert foreign material into the interior of the target particle.

10. The microfabricated particle manipulation system of claim 1, wherein the microfabricated knife edge structure comprises at least one of a knife edge and a point, sufficiently sharp to cut a membrane of the target particle.

11. The microfabricated particle manipulation system of claim 1, wherein the microfabricated knife edge structure comprises a plurality of knife edges, which cut a membrane of the target particle.

12. The microfabricated particle manipulation system of claim 6, wherein the actuator forces material out of the interior of the target particle as a result of deformation.

13. The microfabricated particle manipulation system of claim 1, further comprising a fluidic focusing element, which tends to concentrate the particles toward the center of the microfabricated fluidic channel.

14. The microfabricated particle manipulation system of claim 2, wherein the actuator is formed in a plane parallel to a top surface of the substrate and moves in that plane when actuated.

15. The microfabricated particle manipulation system of claim 2, wherein the foreign material enters in the microfabricated fluidic channel, and enters the particle through a hole pierced in a membrane of the target particle.

16. The microfabricated particle manipulation system of claim 1, further comprising a controller programmed to execute a machine learning algorithm to identify target particles based on information collected in at least one laser interrogation region.

17. The microfabricated particle manipulation system of claim 14,1, wherein the fluid focusing element includes a fluid bend substantially perpendicular to the plane parallel to the surface.

18. The microfabricated particle manipulation system of claim 1, further comprising a compression structure, wherein the compression structure is disposed to restrict the microfabricated fluidic channel and thus apply pressure to the target particles.

19. The microfabricated particle manipulation system of claim 18, wherein the compression structure is magnetically actuated.

20. The microfabricated particle manipulation system of claim 19, wherein in the compression structure also has a piercing structure formed thereon.

21. The microfabricated particle manipulation system of claim 3, further comprising:
a transient pressure generator that supplies a foreign material from the reservoir to the target particles at a time determined by the laser interrogation region.
